(19) United States
(12) Patent Application Publication
(10) Pub. No.: US 2026/0191899 A1
(43) Pub. Date: Jul. 9, 2026

(54) METHOD OF PRODUCING LOCAL NERVE BLOCK BY ADMINISTERING COMPOSITION COMPRISING CYCLODEXTRIN

(71) Applicant: SAOL INTERNATIONAL DEVELOPMENT LTD., Hamilton (BM)

(72) Inventors: John DEVANE, Dublin (IE); David PENAKE, Atlanta, GA (US); Leonard O’MAHONY, Westmeath (IE); Sharon HAMM, Odessa, FL (US); Lopa BHATT, Roswell, GA (US)

(73) Assignee: SAOL INTERNATIONAL DEVELOPMENT LTD., Hamilton (BM)

(21) Appl. No.: 19/316,490

(22) Filed: Sep. 2, 2025

Related U.S. Application Data
(60) Provisional application No. 63/743,460, filed on Jan. 9, 2025.

(51) Int. Cl. A61K 31/724; A61K 9/00; A61K 31/401; A61P 29/00

(52) U.S. Cl. CPC A61K 31/724; A61K 9/0019; A61K 31/401; A61P 29/00

(57) ABSTRACT

Disclosed are methods of treating pain by administering cyclodextrin which is majority unoccupied with a guest compound. The cyclodextrin may also be occupied with an inert molecule or agent as a guest, such as proline. Attenuation of pain treatment due to cyclodextrin may be performed by administration of an inert molecule or agent as a guest, such as proline, either before, after or simultaneously with administration of cyclodextrin.

### CROSS-REFERENCE TO RELATED APPLICATIONS

This application claims priority to U.S. provisional application No. 63/743,460, filed Jan. 9, 2025, the entire contents of which are herein incorporated by reference.

### FIELD

This application relates to, among other things, injectable cyclodextrin formulations and their use.

### BACKGROUND

Cyclodextrins are commonly used as a pharmaceutical excipient, often for improving the solubility of a poorly soluble guest molecule. The mechanism for this generally relies on the ability of cyclodextrin to “encapsulate” the poorly soluble molecule within its “cavity.” This idea can also be referred to as “aggregation” or the formation of an “inclusion complex.” To achieve this end, cyclodextrin is generally included in such compositions iat equivalent molar ratios with the poorly soluble molecule.

When the hydrophobic cyclodextrin cavity is “empty,” i.e., unoccupied by a guest molecule, it has the capacity to cause cell-lysis effects. The mechanism of action of cyclodextrins to disrupt cell membranes is based on a well-established high affinity interaction of cyclodextrins with cholesterol and other lipids in cell membranes (“Use of cyclodextrins to manipulate plasma membrane cholesterol content: Evidence, misconceptions and control strategies,” 2007 Biochimica et Biophysica acta 1768,1311-1324. Zidovetzki et al.). Elegant studies in the literature have described the thermodynamics of these interactions and modelled the extraction of cholesterol from cholesterol monolayers (“Molecular Mechanism of Cyclodextrin Mediated Cholesterol Extraction, 2011 Computational Biology 7 (3), e1002020. Lopez et al.). It has been suggested in the literature that only empty non-occupied cyclodextrins have this cell membrane-lysis effect (“Cyclodextrins: Assessing the impact of Cavity Size, Occupancy and Substitutions on Cytotoxicity and Cholesterol Homeostasis,” 2018 Molecules 23, 1228, Szente et al.).

The present inventors have surprisingly discovered that this mechanism can be applied in vivo to achieve local nerve block and other desirable effects.

### SUMMARY

Provided is a novel composition and method of use where the composition includes only cyclodextrin as the “active” agent, and optionally other pharmaceutically acceptable excipients. The formulation includes cyclodextrin at dose levels generally ranging from about 0.25% to about 50% by weight of the composition, allowing for injection into the area in and/or around a target nerve.

While the mechanism of action of the invention is not fully understood, it is possible that it relies on the ability of the cyclodextrin to extract cholesterol from biological membranes, thereby destabilizing the membranes and causing cell lysis. This cell-lysis effect would damage nerve cells, preventing them from transmitting a nerve impulse, leading to a local nerve block effect.

The cyclodextrin may be any cyclodextrin, such as, for example, hydroxypropyl-β-cyclodextrin (HP-β-CyD). In some embodiments, the concentration of the hydroxypropyl-β-cyclodextrin is from about 5% to about 40% by weight (w/w) of the formulation, such as from about 10% to about 30% by weight (w/w) of the formulation, from about 12% to about 18% by weight (w/w) of the formulation, or from about 14% to about 16% by weight (w/w) of the formulation.

The invention also provides methods for creating a nerve-block in a mammal comprising administering an effective amount of the cyclodextrin formulation, the formulation including at least one cyclodextrin or derivative thereof, and at least one pharmacologically acceptable solvent for administration. In some embodiments, the administering is by injection, and in some embodiments, the administering is by surgical application to an exposed nerve or by topical administration.

The invention also provides methods for treating therapeutic, cosmetic, or tissue lysis condition in a patient in need thereof, by injecting into or applying in proximity of a physiological target area in proximity to or within a nerve, blood vessel, tumour, or tissue growth of the patient an effective amount of a cyclodextrin or derivative thereof, and at least one pharmacologically acceptable solvent for injection.

The invention also provides methods of manufacturing a composition for injection, by combining a cyclodextrin or derivative thereof with a pharmacologically acceptable solvent for injection, and mixing the cyclodextrin or derivative thereof and the pharmacologically acceptable solvent for injection until the cyclodextrin is dissolved.

Additional features and advantages of the present disclosure will be set forth in part in the description that follows, and in part will be apparent from the description, or may be learned by practice of the present disclosure. The objectives and other advantages of the present disclosure will be realized and attained by means of the elements and combinations particularly pointed out in the description and claims.

The foregoing general description and the following detailed description are exemplary and explanatory only to provide a further explanation of the present disclosure and are not restrictive of the scope of the subject matter encompassed by the claims.

### BRIEF DESCRIPTION OF THE DRAWINGS

FIG. 1 shows the stoichiometry of various cyclodextrin (CyD) inclusion complexes.

FIG. 2 shows the dose-responsive effect of hydroxypropyl derivative of β-cyclodextrin (HPBCyD) on RBC-lysis.

FIG. 3 shows the effect of guest occupation on 15% hydroxypropyl derivative of β-cyclodextrin (HPBCyD)-induced RBC-lysis.

FIG. 4 shows the effect of guest occupation on 30% hydroxypropyl derivative of β-cyclodextrin (HPBCyD)-induced RBC-lysis.

FIGS. 5A-5G show images of genicular nerves treated with hydroxypropyl derivative of β-cyclodextrin (HPBCyD).

### DETAILED DESCRIPTION

The particulars shown herein are by way of example and for purposes of illustrative discussion of the various embodiments of the present disclosure only, and provide what is believed to be the most useful and readily understood description of the principles and conceptual aspects of the disclosed subject matter. In this regard, no attempt is made to show details of the disclosed subject matter in more detail than is necessary for a fundamental understanding of the disclosure, the description making apparent to those skilled in the art how the several forms of the disclosure may be embodied in practice.

The following disclosure refers to more detailed embodiments, with occasional reference to the accompanying figures. The disclosed subject matter, however, may be embodied in different forms and should not be construed as limited to the embodiments set forth herein.

Unless otherwise defined, all technical and scientific terms used herein have the same meaning as commonly understood by one of ordinary skill in the art to which this disclosure belongs. The terminology used in the description herein is for describing particular embodiments only and is not intended to be limiting. As used in the specification and claims, the singular forms “a,” “an,” and “the” are intended to include the plural forms as well, unless the context clearly indicates otherwise. Also, the phrases “at least one” and “one or more” are intended to be interchangeable, and their use are not intended to limit the scope of any described or claimed feature preceded by “a,” “an,” and “the” to a singular form.

All publications, patent applications, patents, and other references mentioned herein are expressly incorporated by reference in their entirety, unless otherwise indicated.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by a particular embodiment. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed subject matter are approximations, the numerical values set forth in the specific examples are reported as precisely as possible. Any numerical value, however, inherently contains certain errors necessarily resulting from the standard deviation found in the method used to obtain the value. Every numerical range given throughout this specification includes every narrower numerical range that falls within such broader numerical range, as if such narrower numerical ranges were all expressly written herein.

Reference to compounds in the specification includes esters and salts of such compounds. Thus, even if not explicitly disclosed, such esters and salts are contemplated and encompassed by reference to the compounds themselves.

All percent measurements in this application, unless otherwise stated, are measured by weight based upon 100% of a given sample weight. Thus, for example, 30% represents 30 weight parts out of every 100 weight parts of the sample.

The present disclosure relates, in part, to a composition comprising an active ingredient, and, in some embodiments, an agent for complexing with the active ingredient. The composition may be a pharmaceutical composition.

A “pharmaceutical composition” as used herein means a composition comprising an active ingredient and at least one pharmaceutically acceptable excipient. As used herein, the term “pharmaceutically acceptable excipient” means a compound or ingredient that is compatible with the other ingredients in a pharmaceutical formulation and not injurious to an intended subject when administered in normal or therapeutically effective amounts. As used herein, an “intended subject” includes animals and/or humans. The terms “patient” and “subject” may be used interchangeably.

Suitable excipients are known to those of skill in the art and examples are described, for example, in the Handbook of Pharmaceutical Excipients (Kibbe (ed.), 3rd Edition (2000), American Pharmaceutical Association, Washington, D.C.), and Remington's Pharmaceutical Sciences (Gennaro (ed.), 20th edition (2000), Mack Publishing, Inc., Easton, Pa.), which, for their disclosures relating to excipients and dosage forms, are incorporated herein by reference. Examples of excipients include but are not limited to fillers, extenders, diluents, wetting agents, solvents, emulsifiers, preservatives, absorption enhancers, sustained-release matrices, starches, sugars, microcrystalline cellulose, granulating agents, lubricants, binders, disintegrating agents, coloring agents, release agents, coating agents, sweetening agents, flavoring agents, perfuming agents, antioxidants, plasticizers, gelling agents, thickeners, hardeners, setting agents, suspending agents, surfactants, humectants, carriers, stabilizers, and combinations thereof.

The present disclosure includes a number and variety of components that are contemplated for inclusion in the pharmaceutical formulations. It should be recognized that when the inventors expressly contemplate including such components, they also expressly contemplate excluding such components. Thus, all components disclosed herein are expressly contemplated for exclusion as well.

As used herein, “active ingredient” is any component of the composition intended to furnish pharmacological activity or other direct effect in the diagnosis, cure, mitigation, treatment, or prevention of disease, or to affect the structure or any function of the body of the intended subject. Active ingredients include those components of the composition that may undergo chemical change during the manufacture of the composition and be present in a finished composition in a modified form intended to furnish the specified activity or effect. Active ingredients also include those components of the finished composition that during or after administration of the finished drug product to the intended user may undergo chemical change to a modified form intended to furnish the specified activity or effect. For example, the active ingredient can be a pharmaceutically acceptable salt of the component that furnishes the specified activity or effect. Embodiments disclosed herein include at least one type of cyclodextrin as an active agent, but do not include other active agents. That is, the inventors contemplate excluding all other active agents other than the cyclodextrin component.

As used herein, the term “pharmaceutically acceptable salt” includes salts that are physiologically tolerated by the intended subject. Such salts are typically prepared from an inorganic and/or organic acid. Examples of suitable inorganic acids include hydrochloric, hydrobromic, hydroiodic, nitric, sulfuric, and phosphoric acid. Organic acids may be aliphatic, aromatic, carboxylic, and/or sulfonic acids. Suitable organic acids include formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

As used herein, the term “prevent” or “prevention” in the context of treatment, for example, as in “preventing spasticity/pain” or “prevention of spasticity/pain” refers to a reduction in the spasticity or pain. In other words, as used herein, “prevention” does not require 100% elimination of the symptom.

As noted, cyclodextrin(s) are considered to be the active agent in embodiments of the invention. Cyclodextrins are cyclic oligosaccharides composed of a number of dextrose units of (α-1,4)-linked α-D-glucopyranose. The cyclodextrin structures generally form a hydrophobic/lipophilic central cavity and a hydrophilic outer surface. Cyclodextrins can have up to six, seven, eight, or more units (α-, β-, and γ-CyDs have six, seven, and eight units, respectively). Cyclodextrins are known to interact with hydrophobic drug molecules to form inclusion complexes and can be used to improve aqueous solubility. The structure of the three most common cyclodextrins are shown below for reference and understanding.

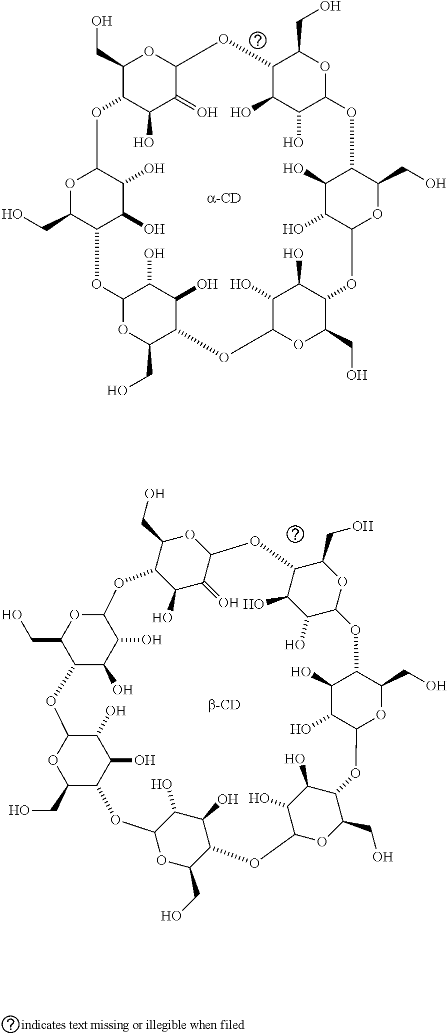

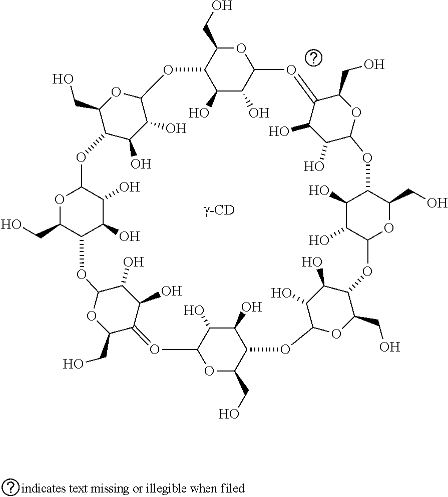

In the pharmaceutical industry, cyclodextrins have mainly been used as complexing agents to increase the aqueous solubility of active substances that are poorly soluble in water in order to increase their bioavailability and to improve stability. In addition, cyclodextrins can be used to reduce or prevent gastrointestinal and ocular irritation, reduce or eliminate unpleasant smells or tastes, prevent drug-drug or drug-additive interactions within a formulation (all these properties are based on reduction of the free drug in solution), or to convert oils and liquid drugs into microcrystalline or amorphous powders. (Brewster ME and Loftsson T (2007) “Cyclodextrins as pharmaceutical solubilizers,” Advanced Drug Delivery Reviews 59:645-666.)

In some embodiments, the alpha, beta, or gamma cyclodextrin (CyD) may be modified or substituted. For instance, the substitution in the alpha, beta, or gamma cyclodextrin may be a hydroxypropyl group, a carboxymethyl group, random methylation, or a sulfobutyl ether group. For β-cyclodextrin, which itself has a relatively low aqueous solubility, substitution of any of the hydrogen bond-forming hydroxyl groups, even by lipophilic functions, results in a dramatic improvement in the aqueous solubility of the derivative. Examples of β-CyD derivatives used as excipients in medicines include, but are not limited to, the sulfobutylether of β-CyD (SBE-β-CyD), the hydroxypropyl derivative of β-CyD (HP—β-CyD or HPBCyD), and the randomly methylated β-CyD (RM-β-CyD). The inventors contemplate the use of these cyclodextrin derivatives, or of any other cyclodextrin derivative that generally functions the same way in the disclosed compositions. While further sections herein will refer HPBCyD, it should be understood that these also contemplate the use of other cyclodextrins.

Each of the cyclodextrins shown above—α-, β-, and γ-CyDs—are contemplated for use in the present invention. Additionally, derivatives of these molecules, examples of which are well known and commercially available, are also contemplated. A skilled person will be able to follow the present disclosure so as to be reproduce the invention using a variety of cyclodextrin derivatives.

In certain embodiments, only one of the noted cyclodextrins or its derivative is used, but combinations of various cyclodextrins may be used as well. Combinations may be useful, for example, to produce a more complex effect profile, if desired. In this regard, each of the different cyclodextrins may have a different ideal dosage range, and by using combinations of different cyclodextrins, more complex treatment effects may be achieved. A skilled person will, with the knowledge of the present disclosure, understand who to make and use such compositions.

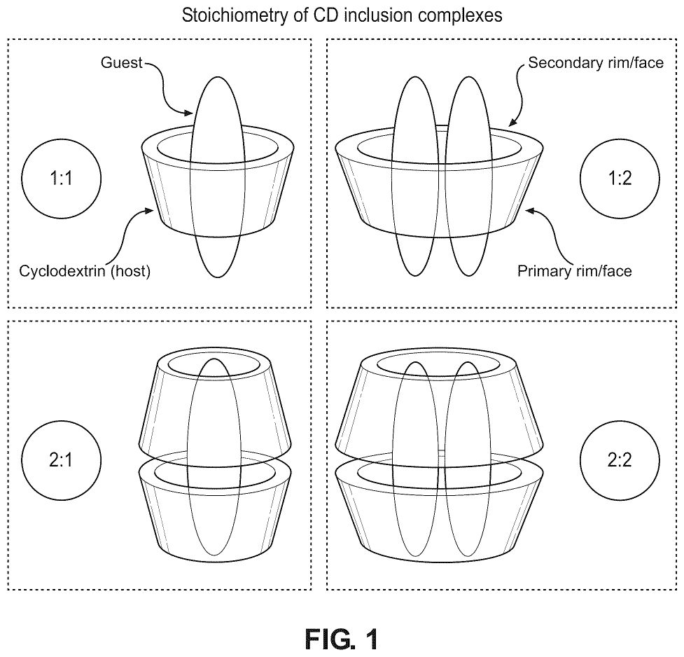

As shown in FIG. 1, the stoichiometry of a cyclodextrin host: guest inclusion complex is most commonly 1:1. However, other stoichiometries such as 1:2, 2:1, and 2:2 are also contemplated.

The common uses of HPBCyD as a pharmaceutical excipient, such as improving the solubility of a poorly soluble guest molecule, generally involves a molar ratio of HPBCyD host to guest drug that is at least equal to 1:1 for a 1:1 or 2:2 stoichiometry, at least equal to 1:2 for a 1:2 stoichiometry, and at least equal to 2:1 for a 2:1 stoichiometry. While further discussion will focus on the most common 1:1 stoichiometry, it will be understood to apply to the other stoichiometries.

For a cyclodextrin (host): guest inclusion complex having a 1:1 stoichiometry, when the guest is in molar excess to the host cyclodextrin, then it can be estimated that the host is fully occupied. Conversely if the guest is only 50% molar equivalent to the cyclodextrin host, then it can be estimated that the host is only 50% occupied.

The inventors discovered that HPBCyD as an empty (or at least majority unoccupied) host molecule has cell-lysis effects and as a result has the ability to have nerve blocking effects if the target nerves are readily accessible to the large, non-lipophilic cyclodextrin molecule.

The molar ratio between the cyclodextrin host and the guest molecule (host:guest) may be, for example, greater than 1:1, greater than 2:1, greater than 3:1, or greater than 7:1. In some embodiments, the composition is substantially free of guest.

The guest molecule may be a pharmacologically active molecule (for example, phenol) or an inert molecule. To form an inclusion complex, the guest must be sufficiently hydrophobic to displace the solvent (for example, water, saline, or buffered saline) from the cyclodextrin cavity. Phenol has been demonstrated to form a 1:1 stoichiometric inclusion complex with HPBCyD.

In some embodiments, the cyclodextrin is at least partially occupied with an inert molecule or agent as a guest. In such a case, the molar ratio between the cyclodextrin host and the guest (host:guest) may be, for example, greater than 1:1, greater than 2:1, greater than 3:1 or greater than 7:1. Examples of the inert molecule or agent can include the amino acid proline, i.e., L-proline.

L-Proline is intermediate in hydrophobicity, and uniquely among the natural amino acids, it does not have an —NH₃ group, but instead the amine nitrogen binds to the side chain forming a 5-membered “ring”. L-Proline has been characterised as forming a 1:1 stochiometric Inclusion Complex with cyclodextrin. (See Kundu et al., “Evidence for complexations of beta-cyclodextrin with some amino acids by h1 NMR, surface tension, volumetric investigations and XRD,” 2017 240, 570-577).

Embodiment compositions of the present invention may be prepared for injection, and in those embodiments, other ingredients in the compositions may be selected based on their compatibility with other components and with the intended use. For example, a vehicle for use in injectable compositions may include water, saline, or any other pharmaceutically acceptable solvent. Water may be most preferable solvent in terms of its ease of use and patient compatibility, but the invention is not limited to the use of Water, or even to aqueous solvents, and non-aqueous solvents are also contemplated.

The concentration of the cyclodextrin may be adjusted as needed by the manufacturer or practitioner, based on observed results and intended use. Because the average molecular weight of the various cyclodextrins and their derivatives varies it will be recognized that appropriate adjustments in concentrations may be required. In the case of HPBCyD typical average molecular weights range between 1380 and 1580 g/mol. Concentrations in the composition may be as low as, for example, 0.25% (2.5 mg/ml or 1.67 mM based on a typical average molecular weight of 1500 g/mol), and may be as high as 120% (1200 mg/ml or 800 mM) based on a typical average molecular weight of 1500 g/mol. The concentration of cyclodextrin and its derivatives in a composition of the present invention is very easily adjusted and modified, and the inventors contemplate all concentrations between these upper and lower values-all possible fractional concentrations are not disclosed, as doing so would be unnecessary. Embodiments may range, for example, from about 15-30% (100-200 mg/ml) or from about 125-175 mg/ml, or from about 140-160 mg/ml, or may be about 150 mg/ml. The amount of the cyclodextrin composition per procedure may range from 0.1 to 20 mL. In some cases, the amount of cyclodextrin composition injected per individual nerve is from 0.1 to 5 mL, such as 1.5 mL per nerve, 2.25 mL per nerve, and 2.7 mL per nerve. As an example, in some cases, 0.33 mL is injected per individual nerve, and three genicular nerves are treated, for a total of 1 mL per procedure. In some other cases, 0.5 to 1 mL is injected per individual nerve, and six genicular nerves are treated, for a total of up to 6 mL per procedure.

Pharmaceutical compositions are typically provided in dosage forms that are suitable for administration to an intended subject by a desired route. Various dosage forms are described below, but are not meant to include all possible choices. One of skill in the art is familiar with the various dosage forms that are suitable for use, as described, for example, in Remington's Pharmaceutical Sciences, which has been incorporated by reference above. The most suitable route in any given case will depend on the nature and severity of the disease and/or condition being prevented, treated, and/or managed. For example, pharmaceutical compositions including the cyclodextrin may be formulated for systemic or localized administration subcutaneously, intramuscularly, perineurally, intraneurally, transdermally, intraarticularly, intrathecally, intravenously, nasally, rectally, intravaginally, intracisternally, and topically.

In some embodiments, the compositions of the invention will be stable at room temperature. As used herein, “stable” means capable of storage without significant alteration in the fundamental composition such that it remains usable as intended. In some embodiments, the compositions will be stable at room temperature; in some embodiments, the compositions will be stable under reduced temperature conditions, such as refrigeration or freezing. In some embodiments, the compositions are stable for one or more days, such as a week or month or more. In some embodiments, the compositions will include one or more additional ingredients to improve stability, such as antioxidants.

The compositions described above can be used in methods for treating, preventing, and/or managing various diseases and/or conditions, comprising administering to a subject or patient in need thereof a therapeutically effective amount of the cyclodextrin. The phrase “therapeutically effective amount” refers to the amount of the active ingredient (e.g., cyclodextrin), which alone or in combination with one or more other ingredients, provides any therapeutic benefit in the prevention, treatment, and/or management of a particular diseases and/or condition. In particular, the compositions may be used in method of pain treatment.

Cyclodextrin is believed to act by impairing nerve conduction nerve cell membrane destabilization, resulting in a nerve block. This can result in medical applications and cosmetic applications. Medical applications can range from local anesthetic to full nerve (motor and sensory) blocks that may be clinically beneficial in conditions such as pain, skeletal muscle spasticity and many others. The same mechanism allows cyclodextrin to be beneficial in cosmetic indications that smooth skin or reduce wrinkles. Finally, the lysing mechanism of cyclodextrin, unrelated to nerve block uses, may allow for clearance of unwanted tissue, tumor lysis, and removal of skin spots.

When being administered for pain treatment, the cyclodextrin may be administered once, or may be repeatedly administered on a regular or irregular basis. For instance, after a first administration, the cyclodextrin may be administered again after 1 month, after 2 months, after 3 months, after 4 months, after 5 months, or after 6 months.

It is also noted that the effect of the invention is expected to be a long-term effect. That is, a single administration is expected to produce a therapeutic effect for at least a day or more, for example, at least a week, one month, two months, three months, four months, five months, six months, seven months, eight months, nine months, ten months, eleven months, or even a year or more.

It should be noted that the clinical compositions in the Examples 3 and 4 are HPBCyD in water and the clinical effects are demonstrated by the changes from baseline pain or spasticity after an injection, and are not compared to a sham procedure control. However, it has been shown in knee pain of osteoarthritis that a sham (saline) procedure itself does not have a meaningful pain reducing effects.

A particular aspect of the current invention relates to perineural injection of motor nerves that innervate major muscles that are subject to altered tone and associated spasticity in patients that have incurred traumatic brain injury, spinal cord injury or stroke. The same may be beneficial to patients with a variety of diseases, including multiple sclerosis, cerebral palsy, ALS, and many others.

A particular aspect of the current invention relates to perineural or generally local injection of cyclodextrin to sensory nerves. The sensory nerve block may be beneficial in multiple pain syndromes such as osteoarthritis of the knee, post-knee replacement pain, cancer pain, and neuralgias.

In some body parts, there is a high degree of variation of the locations of nerves. One example of this is genicular nerves, which surround the knee. It is desirable to provide a treatment which targets smaller, sensory nerves in easily accessible and low risk anatomic locations. Knee pain due to osteoarthritis can be treated in several ways, depending on the severity. The least invasive treatments are weight loss, bracing, use of a cane/assistive device, ice/cryotherapy, heat, oral anti-inflammatory medicines, and targeted exercise, such as exercises of the quadriceps, hip girdle, and core, and cycling or swimming. A more invasive approach is formal physical therapy such as therapy focusing on strength, mobility, gait, ergonomics, pacing, and a graded home exercise program, or joint injections with steroids, hyaluronic acid, or regenerative agents. An even more invasive approach is joint denervation via radiofrequency or chemoneurolysis. An even more invasive approach is surgery, such as arthroplasty. In some embodiments, it is desirable to administer a treatment to the proximity of a sensory nerve and/or to the proximity of a genicular nerve. In these situations, the dosage and administration of the treatment is preferable performed such that the function of a motor nerve is not altered.

The inventors contemplate that a cyclodextrin formulation according to the invention may be used for any indication for which cyclodextrin is approved. Examples of contemplated indications include, but are not limited to, muscle spasticity of any origin or etiology, nerve/neuropathic pain, joint pain, cancer pain, osteoarthritic pain, cosmetic uses, local anesthesia, facet joint pain, discogenic pain, spinal stenosis, Sacro-Illiac joint dysfunction, lumbar spondylosis, migraine related pain, occipital neuralgia, osteosarcoma, soft-tissue sarcoma, ischemic leg pain, post-hip arthroplasty pain, post-knee replacement pain, focal hyperhidrosis, anismus, strabismus, hemifacial spasm, blepharospasm, cervical dystonia, spasmodic dysphonia, sialorrhea, gustatory sweating, facial rejuvenation, temporomandibular pain, peripheral nerve pain, post-herpetic neuralgia, trigeminal neuralgia, chronic migraine, glabellar lines, forehead ryetids, crow's feet, perioral lip lines, overactive bladder, upper limb spasticity, lower limb spasticity, carpal tunnel, fibromyalgia, rectal prolapse, post-surgical opioid reduction, Raynaud phenomenon, hot flashes, genodermatoses, hidradenitis superativa, pompholyx, eccrine nevus, pachynochia congenita, aquagenic keratoderma, androgenetic alopecia, psoriasis, darier disease, alopecia areata, hailey-hailey disease, linear IgA dermatosis, keloids, hypertrophic scars, hereditary spastic paraparesis, herpes zoster pain, radical mastectomy pain, complex regional pain syndrome, spastic diplegia, spastic quadriplegia, Morton's neuroma, post-amputation pain, phantom leg syndrome, hypoxic eschemic cepalopathy of prematurity, pilonidal sinus disease, neuromas, neuralgias, giant gastric leiomyomas, solid tumor devascularization, tumor ablation, herniotic groin pain, varicose veins, skin blemish removal, and wart removal.

The amount of the dose of the active ingredient administered, as well as the dose frequency, will vary depending on the particular dosage form used and route of administration. The amount and frequency of administration will also vary according to the age, body weight, and response of the individual subject or patient. Typical dosing regimens and locations can readily be determined by a competent physician without undue experimentation. It is also noted that the clinician or treating physician will know how and when to interrupt, adjust, or terminate therapy in conjunction with individual subject or patient response.

Changes to the efficacy and non-targeted tissue damage may be influenced by increases or decreases in blood flow. The inventors contemplate that an increase in blood flow of local, non-targeted tissue may further enhance a reduction in tissue damage including but not limited to reduction in damage to the skin or skeletal muscle. This may be important for any application but particularly those in areas of sensitive or thin tissues such as the face, head, hand, feet or pubic regions for local therapeutic or cosmetic interventions. Changes in local blood flow could be created via any number of applications ranging from surface heating or cooling of the local area to injection of medications that change local blood flow.

The cyclodextrin formulation may be combined with dyes or other agents that support visualization of the site of application. Injectable versions of cyclodextrin may be localized with ultrasound, electronic stimulation, fluoroscopy or other potential visualization techniques. Dyes may also support visual localization of the cyclodextrin following application to the skin or direct injection while using cameras to support procedures.

The localization of the application, via injection or otherwise, may be desired to be more targeted in areas with complex soft-tissue structures in or near the targeted nerve or vein. In other potential uses, particularly those that are in close proximity to tissues such as bone like joint injections and benefit from blocking multiple nerves may benefit from the spreading effect that comes following injection of the cyclodextrin formulation. A physician or other skilled clinician, having read the present disclosure, will be able to readily determine the appropriate site of administration, as well as the dosing and frequency thereof. In this regard, it is not critical that the composition be injected directly into a nerve (as doing so can sometimes result in pain); rather, the composition can be injected or otherwise applied in a manner that it is applied to rather than into the nerve. Stated differently, the invention contemplates the application of the compositions directly or indirectly to nerve tissue.

This formulation may be used intravenously or intravascularly to cause localized vein sclerosis that may be useful in treatments of tumors, cancer, or cosmetic applications like varicose veins.

Applications of the cyclodextrin formulation on moles or other skin spots may create a localized lysis to remove unwanted spots in a localized and non-surgical manner. A cyclodextrin formulation may also be used for topical use on a broader area of skin.

Typical single treatment, in potentially multiple injections or administrations, maximum doses of cyclodextrin may be up to 5 grams per treatment exposure. The formulation may allow for the use of cyclodextrin beyond 5 grams of daily systemic exposure and may provide for greater flexibility in frequency of exposure.

As described above, unoccupied cyclodextrin or majority-unoccupied cyclodextrin may be used in a method of treating pain, for example by administering the cyclodextrin to the pain site. However, it is also possible to attenuate this treatment by administering an inert guest molecule such as proline before, after or simultaneously with the cyclodextrin. The inert guest molecule can be administered via any systemic or localized means, such as subcutaneously, intramuscularly, perineurally, intraneurally, transdermally, intraarticularly, intrathecally, intravenously, nasally, rectally, intravaginally, intracisternally, and topically.

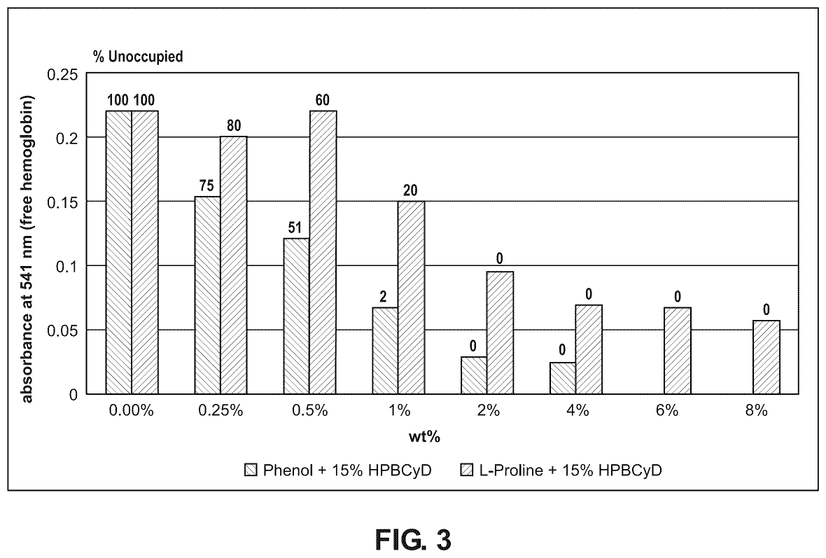

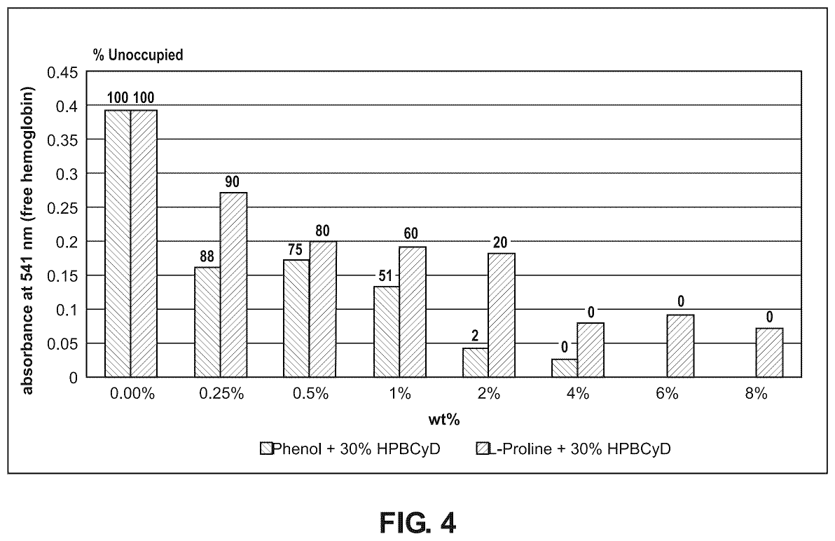

As shown in FIGS. 3 and 4 for example, cyclodextrin which is fully occupied with proline (or another inert molecule) has a reduced cytolytic effect. As such, fully occupied cyclodextrin will have a reduced pain treatment effect as compared to fully unoccupied or majority unoccupied cyclodextrin. Therefore, a clinician may choose to use a standard dose of cyclodextrin and modulate the effect of the cyclodextrin by providing the appropriate concentration of, for example, proline (or other inactive occupying agent) to attenuate the effect of the standard dose of cyclodextrin. In such a case, the proline may be administered simultaneously or prior to administration of the cyclodextrin.

Alternatively, a clinician may administer cyclodextrin first, and then administer the proline (or another inert molecule) at a desired time thereafter, such as after 1 hour, 4 hours, 12 hours, 24 hours or 48 hours, for example. This subsequent administration of proline will slow or halt the effect of cyclodextrin. In one embodiment, the inert molecule can be administered topically to the ear, such as by ear drops. In such a case, cyclodextrin is administered systemically for a particular condition, such as Neiman-Pick Type C (NPC) Syndrome for example. However, this systemic administration of the cyclodextrin is known to cause hearing toxicity when the administration is in the proximity to the ear. In such a case, proline drops may be used to interact with and thereby occupy the cyclodextrin to at least attenuate the undesirable side effect.

### EXAMPLES

### Example 1: In Vitro Experiments

An in vitro red blood cell (RBC)-lysis study was performed. RBC-lysis is a well-established in vitro method to assess the effects of drugs on cell membranes. Using absorbance at 541 nm (free hemoglobin) as a measure of cell lysis, a set of in vitro experiments were conducted. Molar concentrations of HPBCyD and estimated % unoccupied are based on the average molecular weight of the specific HPBCyD batch used in these experiments (1387 g/mol) and a density of 1.05.

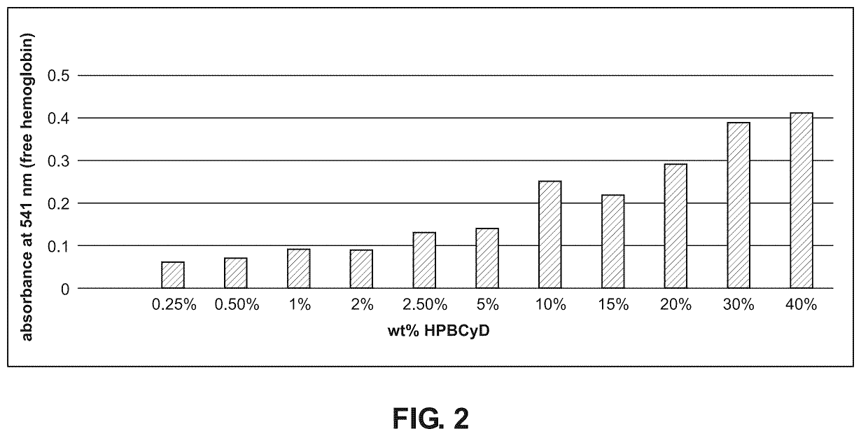

In FIG. 2, 200 μl of HPBCyD (0.25-40%) was added to 25 μl suspended RBC in a total 1 ml volume of PBS (Phosphate Buffered saline). HPBCyD shows a graded concentration-dependent RBC-lysis effect confirming that HPBCyD has the potential to be nerve cell lytic and potentially nerve-blocking clinically.

Both 15% HPBCyD (approx. 50% maximal RBC-lysis effect) and 30% HPBCyD (maximal RBC-lysis effect) were selected as representative concentrations for additional experiments. In FIGS. 3 and 4, 200 μl of either phenol (0.25-4%) or proline (0.25-8%) and either 15% or 30% HPBCyD were added to the suspended RBC in a total 1 ml PBS volume. As shown in FIGS. 3 and 4, when the host HPBCyD is only minimally occupied by phenol or proline, substantial cell lysis is observed.

However, as the molar concentration of phenol or proline to HPBCyD increases to 1:1 and the HPBCyD is fully occupied (i.e., 0% unoccupied), the cell lytic effects are lost. It is noted that in FIGS. 3 and 4, data corresponding to the combination of 15% or 30% HPBCyD with higher phenol concentrations of 6% and 8% are not included. This is because phenol itself has potent cytolytic effects at these higher concentrations and confound the reduced cell lytic effects of occupied HPBCyD. As such, for clarity, this data is omitted.

### Example 2: Spasticity Study

A single ascending dose escalation study was performed in patients with limb spasticity.

The study included four cohorts treated with 15% HPBCyD in water, as in Table 1 below:

TABLE 1

| | | | | | | | | Total |
|---|---|---|---|---|---|---|---|---|
| | | | | | | Mean | | nerves |
| Study Design | | | | | | Dose | | treated |
| Cohort 1 | 2 patients | 1 | nerve | 1 | mL | 1.00 | mL | 2 |
| Cohort 2 | 2 patients | 3 | nerves | 2-3 | mL | 2.50 | mL | 3 |
| Cohort 3 | 2 patients | 4 | nerves | 4-9 | mL | 4.50 | mL | 6 |
| Cohort 4 | 2 patients | 6 | nerves | 10-16 | mL | 14.00 | mL | 10 |

The primary end point was an assessment of the overall safety of the single treatment exposure. The secondary endpoints were: (1) assessment of efficacy measures associated with spasticity to include quantified change from baseline of Modified Ashworth Score (MAS), Disability Assessment Scale (DAS) and Modified DAS Lower Limbs, Goal Attainment Scale (GAS), Clinical Global Impression of Change (CGI-C) as assessed by Investigator and patient, spasticity related pain via Numeric Rating Scale (NRS) and the Tardieu scale score. Patients were evaluated on days 1, 7, 14, 28, 56, 84, 112, and 168, plus-or-minus 1-3 days.

The following were selection inclusion criteria for the spasticity study:

(1) spasticity based on MAS of greater than or equal to 2 in the specified limbs and muscle groups.

(2) Patients who are diagnosed with spasticity of cerebral or spinal origin that can be treated with chemodenervation of the approved cohort-specific nerves.

(3) Patients who have had spasticity for greater than 6 months and have not had a significant change in their spasticity during recent evaluations or changes in dosing of spasticity medication in the 2 weeks prior to the Screening visit.

(4) Male or female patients between 18-80 years of age. For study Cohorts 1 and 2, patients must have a total body weight of ≥40 kg. For study Cohorts 3 and 4, patients must have a total body weight of ≥45.5 kg. Investigator consideration should be given to the appropriateness of the patient body composition to enable accurate targeting of the identified nerve(s) for treatment.

(5) Female patients who are not pregnant, not potentially childbearing, or willing to use acceptable contraceptive methods throughout the study and for 30 days after the last study drug administration.

(6) Patients who are mentally coherent and responsive to communication, and instructional command and have the ability to communicate proactively as defined by a Mini Mental State Examination (MMSE) score of 18 or greater. Patients can be enrolled into the study with or without the assistance of a caregiver based upon Investigator's discretion. Patients who are cognitively compromised will be required to have a legally authorized representative consent for the study on their behalf.

(7) Patients who have a positive response to baseline screening of 2-8 mL lidocaine 1% solution administration.

(8) Patients with no prior history or significant trauma to the injected limbs.

The following were selection exclusion criteria for the spasticity study:

(1) Muscle contractures of the targeted upper or lower limb joints or any challenge that causes severe restriction of range of motion of targeted joints. Contractures will be identified by the Investigator following the lidocaine block by assessing the response.

(2) Patients with severe muscle atrophy in the targeted limbs.

(3) Patients who have had long-term treatments to block nerves (alcohol, phenol, etc.) that will be targeted in the study within 6 months of the Screening visit. Time since last nerve block treatment and anticipated conduct of the Screening Visit will be based upon Investigator discretion for any given patient being considered for study participation.

(4) Patients currently on intrathecal or oral baclofen therapy for spasticity who have not been on a stable dose regimen for at least 2 weeks prior to the Screening Visit or who would need to have significant dose adjustments at any time point during study participation.

(5) Patients who have received any botulinum toxin in the targeted muscle groups for the nerve block treatment within 6 months of the Screening Visit. Note: patients will not be able to receive any botulinum toxin injections to the targeted muscle group from the Screening Visit to the End of Study visit.

(6) Patients with bleeding disorders or who are being treated with anticoagulants.

(7) Patients with severe hepatic or renal impairment that, in the judgement of the Principal Investigator, will preclude participation in the study.

(8) Female patients who are pregnant or planning to become pregnant during the duration of the study.

(9) Female patients who are breast-feeding.

(10) Patients taking any of the following concurrent medications/over-the counter products: Probenecid or other OAT3 inhibitors, or Inhibitors of CYP2E1, such as disulfiram.

Muscle groups and nerves injected in the spasticity study were as follows: shoulder adductors/shoulder internal rotators (e.g., pectoral lateral nerve; pectoral medial nerve); elbow flexors/elbow extensors (e.g., musculocutaneous nerve; radial nerve); wrist extensors/wrist flexors (e.g., radial nerve; medial nerve); finger flexors/forearm pronators/MCP flexors (e.g., median nerve); thumb adductors (e.g., median nerve); hip adductors/hip internal rotators (e.g., obturator nerve); knee flexors/knee extensors (e.g., femoral nerve; sciatic nerve); ankle plantar flexors/ankle inverters (e.g., tibial nerve); and toe flexors (e.g., tibial nerve).

It was surprisingly found that rapid improvements in the MAS score were seen immediately on Day 1, and were sustained for up to 168 days, which was the full extent of measurement. See Table 2, below.

TABLE 2

| | Change in MAS | Change in MAS | Change in MAS |
|---|---|---|---|
| Day | score (all nerves) | score (upper limbs) | score (lower limbs) |
| 0 | 0 | 0 | 0 |
| 7 | −1.19 | −1.24 | −0.5 |
| 14 | −0.93 | −1.03 | −0.25 |
| 28 | −0.74 | −0.67 | −0.3 |
| 56 | −1.0 | −1.38 | −0.1 |
| 84 | −0.68 | −0.71 | 0 |
| 168 | −0.33 | −0.3 | 0.2 |

As noted above, a sham procedure is not believed to have a cytolytic effect. As such, it is concluded that the improvements seen are purely due to the 15% HPBCyD administered.

### Example 3: Osteoarthritis Knee Pain Study

A single-ascending dose escalation study was performed to assess the safety and efficacy of 15% HPBCyD in water in knee pain associated with osteoarthritis.

The study included four cohorts, as in Table 3 below:

TABLE 3
Study Design (n = 124)
3:1 randomization

| | | | |
|---|---|---|---|
| Cohort 1 | 2 patients | 3 nerves | 1 mL |
| Cohort 2 | 2 patients | 6 nerves | 3 mL |
| Cohort 3 | 2 patients | 6 nerves | 6 mL |
| Cohort 4 | 27 patients | 6 nerves | 6 mL |

The primary endpoints of the study were (1) assessing the overall safety through the final study visit at 6 months and (2) improvement in weight-bearing pain in comparison at month 3. The secondary endpoint of the study was improvement in pain and function at month 3. The study explored the improvement in pain and function at month 6 and the use of concomitant pain medication, and assessed procedure-associated pain.

Patients were evaluated on days 2, 7, 14, 28, 56, 84, and 168, plus-or-minus 1-14 days.

The following were selection inclusion criteria for the knee pain study:

(1) Male or female patients 35 years of age or older, with a Body Mass Index (BMI) of
18.0 to 40.0 kg/m2 and a total body weight of ≥50.0 kg for males and ≥45.5 kg for females.

(2) Patients with chronic knee pain resulting from osteoarthritis for greater than 6 months
before study screening that interferes with functional activities (e.g., ambulation, prolonged standing, etc.).

(3) Patients currently experiencing continued pain despite receiving at least 3 months of conservative treatments, inclusive of activity modification, home exercise, protective weight bearing, and/or use of analgesics (e.g., acetaminophen or NSAIDs).

(4) Patients with a baseline average pain score while walking of ≥6 on an 11-point
NRS scale over the past 24 hours for the index knee at Screening. The baseline average pain score is computed by finding the mean of the daily NRS pain intensity scores reported during the 7-day run-in period.

(5) Patients with Kellgren-Lawrence Grade 2 (mild) or Grade 3 (moderate) radiologically confirmed osteoarthritis (via x-ray/MRI/CT) within 6 months of study screening.

(6) Patients with a baseline KOOS pain subscale score≤67 in the index knee.

(7) Patients who have a positive response to a single genicular nerve block of the index knee using 1.5-3 mL of lidocaine 2% solution administration to the genicular nerves planned for treatment in each cohort (0.5 mL per nerve). A positive response to the qualifying lidocaine prognostic block is defined as ≥80% pain reduction within 60 minutes of receiving the injections and ≥80% pain relief for a duration of at least 60 minutes as documented on a standardized 6-hour pain log.

The following were selection exclusion criteria for the knee pain study:

(1) Patients with evidence of inflammatory arthritis (e.g., rheumatoid arthritis) or any other systemic inflammatory condition (e.g., gout, pseudogout).

(2) Current diagnosis of fibromyalgia.

(3) Patients with evidence of neuropathic pain affecting the index knee.

(4) Patients who have received an intra-articular steroid injection into the index knee
within 90 days of study screening.

(5) Patients who have received a hyaluronic acid injection, PRP, stem cell or arthroscopic
debridement/lavage injection into the index knee within 180 days of study screening.

(6) Patients who have received prior radiofrequency ablation or any other neurolytic
procedure of the genicular nerves of the index knee within 1 year of study screening.

(7) Patients who have received prior partial, resurfacing, or total knee arthroplasty of the
index knee (residual hardware).

(8) Patients with clinically significant valgus/varus deformities or evidence of pathology
(other than osteoarthritis of the knee) that materially affects gait or function of the index knee or is the underlying cause of the knee pain and/or functional limitations.

(9) Patients with chronic pain are associated with significant psychosocial dysfunction.

(10) Patients with a Patient Health Questionnaire-9 (PHQ-9) score of >10 (indicative of a state of moderate depression). Patients are currently prescribed opioid medications at a dose of >50 daily morphine equivalents.

(11) Patients with a documented history or evidence of alcohol or drug abuse within 1 year of study screening

Work by Tran et al. (Reg Anesth Pain Med., 2018) enhanced the understanding of potential neuroanatomical targets by utilizing fundamental pain management techniques to target smaller, sensory, genicular nerves in easily accessible and low risk anatomic locations. These nerves include the nerve to vastus intermedius, the nerve to vastus lateralis, the superior lateral genicular nerve (SLGN), the terminal articular branch common fibular nerve (TABCFN), the inferior lateral genicular nerve (ILGN), the superior medial genicular nerve (SMGN), the inferior lateral genicular nerve (IMGN), the common fibular nerve, the recurrent fibular nerve, the nerve to vastus medialis (NVM), and the intrapatellar branch of the saphenous nerve (IPBSN).

Other potential target nerves for the treatment disclosed herein include, but are not limited to, the tibial nerve (including superior and inferior branches thereof), the posterior division of the obturator nerve, the anterior division of the obturator nerve, the common fibular and sciatic nerves (including articular, anterior, posterior branches thereof).

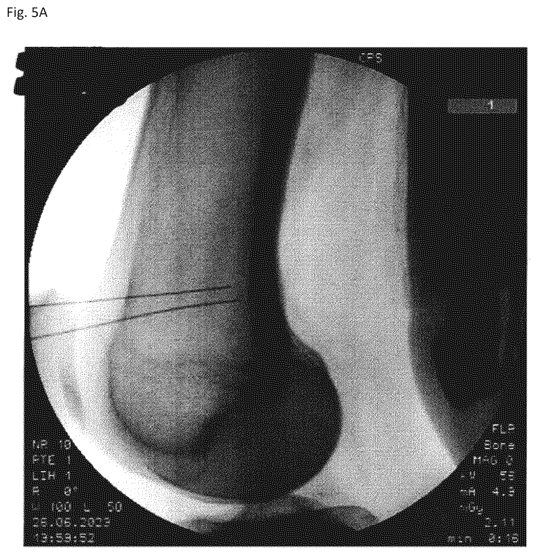

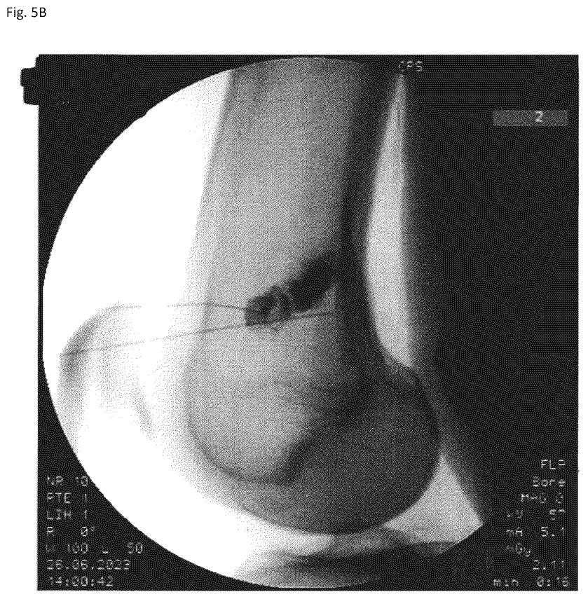

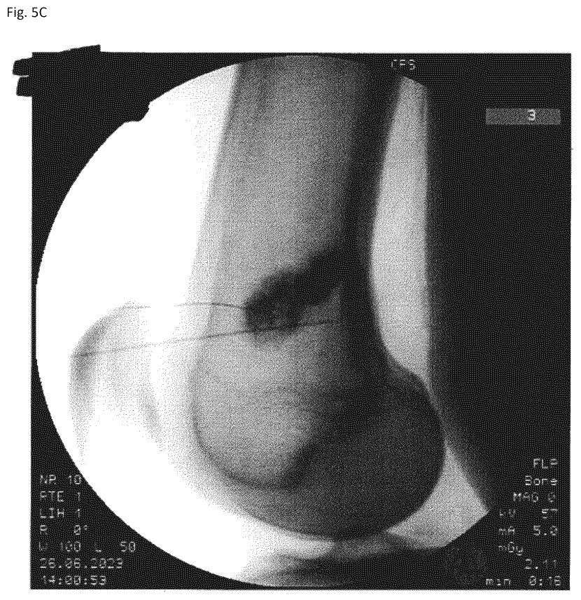

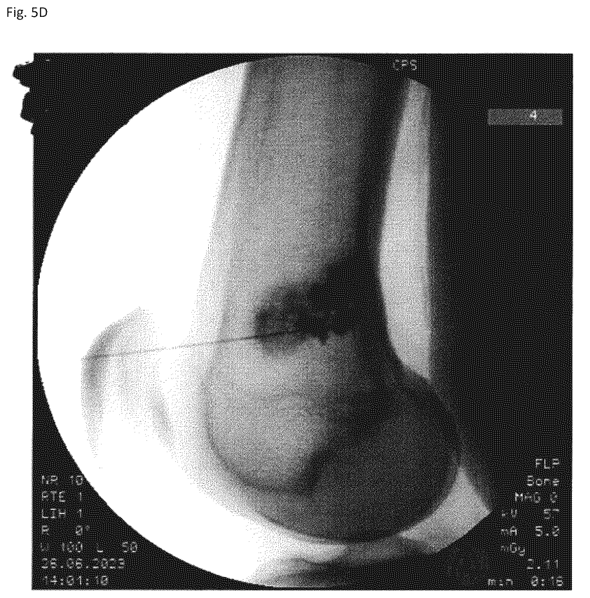

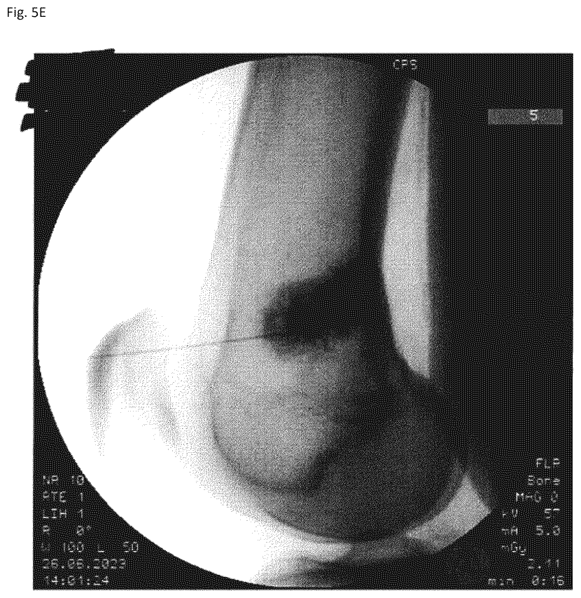

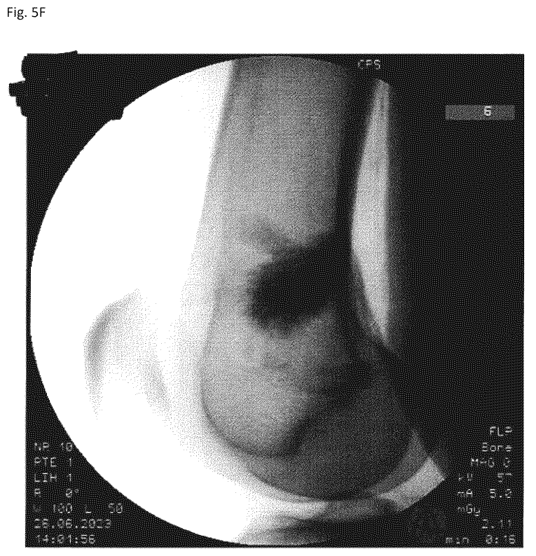

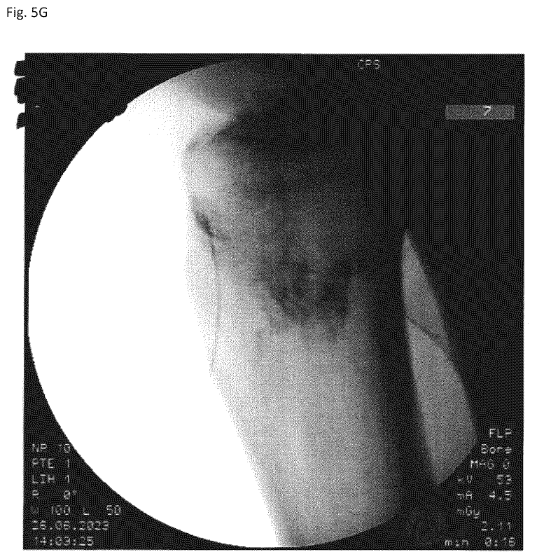

FIGS. 5A-5G are sequential photographs in one patient showing the injection of a fluorescent dye to ensure that the injection needle was correctly positioned. After confirmation of positioning, the 15% HPBCyD solution was injected.

The procedure was found to be well-tolerated. The average treatment time was approximately 10 minutes, with 25% of all treatments completed in 7 minutes or less.

Overall, the treatment was considered well-tolerated with no therapy or program limiting events.

However, it was surprisingly found that, patients receiving the 15% HPBCyD solution showed marked pain relieving effects. Table 4 below shows the response rate to 15% HPBCyD. Table 5 below shows the NRS (pain intensity while walking) score in response to HPBCyD treatment by Week Intent To Treat (ITT) analysis population. Table 6 below shows a study extension showing an analysis of change from baseline of NRS (average pain intensity) at week 48 (12 months) ITT analysis population.

TABLE 4

| | | Percentage with >50% pain |
|---|---|---|
| Week | N (29) | reduction |
| 1 | 17 | 58.6% |
| 2 | 17 | 58.6% |
| 4 | 18 | 62.1% |
| 8 | 15 | 51.7% |
| 12 | 17 | 58.6% |
| 24 | 18 | 62.1% |

TABLE 5

| Week | Mean (N = 29) |
|---|---|
| Baseline | 7.4 |
| Week 1 | 3.6 |
| Week 2 | 3.3 |
| Week 4 | 3.6 |
| Week 8 | 3.6 |
| Week 12 | 3.5 |
| Week 24 | 3.4 |

TABLE 6

| Week | Mean (N = 12) |
|---|---|
| Week 1 | −3.55 |
| Week 2 | −3.97 |
| Week 4 | −4.14 |
| Week 8 | −4.15 |
| Week 12 | −4.55 |
| Week 24 | −4.08 |
| Week 36 | −3.11 |
| Week 48 | −3.64 |

As noted above, a sham procedure (saline) is not believed to have a cytolytic effect. As such, it is concluded that the improvements seen are purely due to the 15% HPBCyD administered.

The present disclosure includes any combination of these various features or embodiments above and/or below as set forth in sentences and/or paragraphs. Any combination of disclosed features herein is considered part of the present disclosure and no limitation is intended with respect to combinable features.

Applicant specifically incorporates the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

Other embodiments of the present disclosure will be apparent to those skilled in the art from consideration of the present specification and practice of the present disclosure disclosed herein. It is intended that the present specification and examples be considered as exemplary only with a true scope and spirit of the disclosure being indicated by the following claims and equivalents thereof.

### REFERENCES

1. Brewster ME and Loftsson T (2007) Cyclodextrins as pharmaceutical solubilizers. Advanced Drug 143, Delivery Reviews 59:645-666.
2. Szente, Lajos, Szejtli J and Kis, G. L. Spontaneous Opalescence of Aqueous γ-Cyclodextrin Solutions: Complex Formation or Self-Aggregation? Journal of Pharmaceutical Sciences 1998: 87 (6): 778-781.
3. Kundu Mitali, Subhadeep Saha, Mahendra Nath Roy, Evidences for complexations of β-cyclodextrin with some amino acids by 1H NMR, surface tension, volumetric investigations and XRD, Journal of Molecular Liquids, Volume 240, 2017, Pages 570-577
4. López CA, de Vries AH, Marrink SJ. Molecular mechanism of cyclodextrin mediated cholesterol extraction. PLOS Comput Biol. 2011 March; 7 (3): e1002020. doi: 10.1371/journal.pcbi.1002020. Epub 2011 Mar. 24. PMID: 21455285; PMCID: PMC3063748.
5. Tran J, Peng PWH, Lam K, Baig E, Agur AMR, Gofeld M. Anatomical Study of the Innervation of Anterior Knee Joint Capsule: Implication for Image-Guided Intervention. Reg Anesth Pain Med. 2018 May; 43 (4): 407-41

What is claimed is:

1. A method of treating pain in a subject in need of treatment, comprising:
injecting the patient with a composition comprising a majority unoccupied cyclodextrin locally to a sensory nerve of a pain site or source, the cyclodextrin being majority unoccupied with a guest compound.

2. The method of claim 1,
wherein the majority unoccupied cyclodextrin is a host,
wherein the compound is a guest, and
wherein
(a) in the case of a 1:1 or 2:2 inclusion complex stoichiometry, a molar ratio between the host and the guest is greater than 1:1,
(b) in the case of a 1:2 inclusion complex stoichiometry, the molar ratio between the host and the guest is greater than 1:2, or
(c) in the case of a 2:1 inclusion complex stoichiometry, the molar ratio between the host and the guest is greater than 2:1.

3. The method of claim 2,
(a) in the case of the 1:1 or 2:2 inclusion complex stoichiometry, a molar ratio between the host and the guest is greater than 2:1,
(b) in the case of the 1:2 inclusion complex stoichiometry, the molar ratio between the host and the guest is greater than 2:2, or
(c) in the case of the 2:1 inclusion complex stoichiometry, the molar ratio between the host and the guest is greater than 4:1.

4. The method of claim 2,
(a) in the case of the 1:1 or 2:2 inclusion complex stoichiometry, a molar ratio between the host and the guest is greater than 4:1,
(b) in the case of the 1:2 inclusion complex stoichiometry, the molar ratio between the host and the guest is greater than 4:2, or
(c) in the case of the 2:1 inclusion complex stoichiometry, the molar ratio between the host and the guest is greater than 8:1.

5. The method of claim 2, wherein
(a) in the case of the 1:1 or 2:2 inclusion complex stoichiometry, a molar ratio between the host and the guest is greater than 7:1,
(b) in the case of the 1:2 inclusion complex stoichiometry, the molar ratio between the host and the guest is greater than 7:2, or
(c) in the case of the 2:1 inclusion complex stoichiometry, the molar ratio between the host and the guest is greater than 14:1.

6. The method of claim 1, wherein the composition is substantially free of phenol.

7. The method of claim 1, wherein the majority unoccupied cyclodextrin is alpha, beta, or gamma cyclodextrin.

8. The method of claim 7, wherein the cyclodextrin is substituted alpha, beta, or gamma cyclodextrin.

9. The method of claim 8, wherein the substitution is a hydroxypropyl group.

10. The method of claim 8, wherein the substitution is a carboxymethyl group.

11. The method of claim 8, wherein the substitution is random methylation.

12. The method of claim 8, wherein the substitution is a sulfobutyl ether group.

13. The method of claim 1, wherein the majority unoccupied cyclodextrin is majority unoccupied hydroxypropyl cyclodextrin.

14. The method of claim 13, wherein the majority unoccupied hydroxypropyl cyclodextrin is majority unoccupied hydroxypropyl beta cyclodextrin.

15. The method of claim 13, wherein the majority unoccupied hydroxypropyl cyclodextrin is majority unoccupied hydroxypropyl alpha cyclodextrin.

16. The method of claim 13, wherein the majority unoccupied hydroxypropyl cyclodextrin is majority unoccupied hydroxypropyl gamma cyclodextrin.

17. The method of claim 1, wherein the subject is a subject with symptoms of pain associated with osteoarthritis.

18. The method of claim 1, wherein the pain site or source is a knee.

19. The method of claim 1, wherein the pain site or source is a shoulder.

20. The method of claim 1, wherein the pain site or source is a vertebrae.

21. The method of claim 1, wherein the pain site or source is a joint.

22. The method of claim 21, wherein the joint is selected from the group consisting of a shoulder, elbow, wrist, hip, knee, ankle, finger, or toe.

23. The method of claim 1, wherein the composition is locally injected such that the composition is delivered to the proximity of a sensory nerve.

24. The method of claim 1, wherein the composition is locally injected such that the composition does not alter the function of a motor nerve.

25. The method of claim 1, wherein the composition is locally injected such that the composition is delivered to the proximity of a genicular nerve.

26. The method of claim 1,
wherein the composition comprises the majority unoccupied hydroxypropyl cyclodextrin at a concentration of greater than 13.33 mM, and
wherein 0.1 mL to 20 mL of the composition is injected.

27. The method according to claim 26, wherein the composition comprises the majority unoccupied hydroxypropyl cyclodextrin at a concentration of 100 mM to 200 mM.

28. The method of claim 1, wherein the composition consists essentially of the majority unoccupied hydroxypropyl cyclodextrin and a pharmaceutically acceptable carrier.

29. The method of claim 28, wherein the majority unoccupied hydroxypropyl cyclodextrin is majority unoccupied hydroxypropyl beta cyclodextrin and wherein the pharmaceutically acceptable carrier is saline, water for injection, phosphate buffered saline (PBS), or water.

30. The method of claim 1, wherein the injection is perineural.

31. The method of claim 1, wherein the injection is intramuscular.

32. The method of claim 1, wherein the injection is subcutaneous.

33. The method of claim 1, wherein the injection is intradermal.

34. The method of claim 1, wherein the injection is intravenous.

35. The method of claim 1, wherein the injection is intraarticular.

36. The method of claim 1, wherein the injection is repeated after at least 1 month.

37. The method of claim 1, wherein the injection is repeated after at least 2 months.

38. The method of claim 1, wherein the injection is repeated after at least 3 months.

39. The method of claim 1, wherein the injection is repeated after at least 4 months.

40. The method of claim 1, wherein the injection is repeated after at least 5 months.

41. The method of claim 1, wherein the injection is repeated after at least 6 months.

42. A method of treating pain in a subject in need of treatment, comprising:
injecting the patient with a composition comprising cyclodextrin as a host and an inert molecule or agent as a guest locally to a sensory nerve of a pain site or source,
wherein the inert molecule or agent does not cause membrane cell lysis in a red blood cell (RBC) test.

43. The method of claim 42,
wherein
(a) in the case of a 1:1 or 2:2 inclusion complex stoichiometry, a molar ratio between the host and the guest is greater than 1:1,
(b) in the case of a 1:2 inclusion complex stoichiometry, the molar ratio between the host and the guest is greater than 1:2, or
(c) in the case of a 2:1 inclusion complex stoichiometry, the molar ratio between the host and the guest is greater than 2:1.

44. The method of claim 43,
(a) in the case of the 1:1 or 2:2 inclusion complex stoichiometry, a molar ratio between the host and the guest is greater than 2:1,
(b) in the case of the 1:2 inclusion complex stoichiometry, the molar ratio between the host and the guest is greater than 2:2, or
(c) in the case of the 2:1 inclusion complex stoichiometry, the molar ratio between the host and the guest is greater than 4:1.

45. The method of claim 43,
(a) in the case of the 1:1 or 2:2 inclusion complex stoichiometry, a molar ratio between the host and the guest is greater than 4:1,
(b) in the case of the 1:2 inclusion complex stoichiometry, the molar ratio between the host and the guest is greater than 4:2, or
(c) in the case of the 2:1 inclusion complex stoichiometry, the molar ratio between the host and the guest is greater than 8:1.

46. The method of claim 43,
(a) in the case of the 1:1 or 2:2 inclusion complex stoichiometry, a molar ratio between the host and the guest is greater than 7:1,
(b) in the case of the 1:2 inclusion complex stoichiometry, the molar ratio between the host and the guest is greater than 7:2, or
(c) in the case of the 2:1 inclusion complex stoichiometry, the molar ratio between the host and the guest is greater than 14:1.

47. The method of claim 42, wherein the inert molecule or agent is proline.

48. The method of claim 42, wherein the composition is substantially free of phenol.

49. The method of claim 42, wherein the cyclodextrin is substituted cyclodextrin.

50. The method of claim 49, wherein the substituted cyclodextrin is methyl substituted.

51. The method of claim 49, wherein the substituted cyclodextrin is sulfo substituted.

52. The method of claim 42, wherein the cyclodextrin is hydroxypropyl cyclodextrin.

53. The method of claim 52, wherein the hydroxypropyl cyclodextrin is hydroxypropyl beta cyclodextrin.

54. The method of claim 52, wherein the hydroxypropyl cyclodextrin is hydroxypropyl alpha cyclodextrin.

55. The method of claim 52, wherein the hydroxypropyl cyclodextrin is hydroxypropyl gamma cyclodextrin.

56. The method of claim 42, wherein the subject is a subject having osteoarthritis.

57. The method of claim 42, wherein the pain site or source is a knee.

58. The method of claim 42, wherein the pain site or source is a shoulder.

59. The method of claim 42, wherein the pain site is a vertebrae.

60. The method of claim 42, wherein the pain site is a joint.

61. The method of claim 60, wherein the joint is selected from the group consisting of a shoulder, elbow, wrist, hip, knee, ankle, finger, or toe.

62. The method of claim 42, wherein the composition is locally injected such that the composition is delivered to the proximity of a sensory nerve.

63. The method of claim 42, wherein the composition is locally injected to target a sensory nerve such that the composition does not alter the function of any motor nerve.

64. The method of claim 42, wherein the composition is locally injected such that the composition is delivered to the proximity of a genicular nerve.

65. The method of claim 42,
wherein the composition comprises the unoccupied hydroxypropyl cyclodextrin at a concentration of 10 mM to 600 mM, and
wherein 0.1 mL to 20 mL of the composition is injected.

66. The method of claim 42, wherein the composition consists essentially of the cyclodextrin, the proline, and a pharmaceutically acceptable carrier.

67. The method of claim 42, wherein the cyclodextrin is hydroxypropyl beta cyclodextrin and wherein the pharmaceutically acceptable carrier is saline, water for injection, phosphate buffered saline (PBS), or water.

68. The method of claim 42, wherein the injection is perineural.

69. The method of claim 42, wherein the injection is intramuscular.

70. The method of claim 42, wherein the injection is subcutaneous.

71. The method of claim 42, wherein the injection is intradermal.

72. The method of claim 42, wherein the injection is intraarticular.

73. The method of claim 42, wherein the injection is intravenous.

74. The method of claim 42, wherein the injection is repeated after at least 1 month.

75. The method of claim 42, wherein the injection is repeated after at least 2 months.

76. The method of claim 42, wherein the injection is repeated after at least 3 months.

77. The method of claim 42, wherein the injection is repeated after at least 4 months.

78. The method of claim 42, wherein the injection is repeated after at least 5 months.

79. The method of claim 42, wherein the injection is repeated after at least 6 months.

80. A method of attenuating a cytolytic effect of cyclodextrin in a subject receiving cyclodextrin treatment, having previously received cyclodextrin treatment, or planned to receive cyclodextrin treatment, comprising
administering an inert molecule or agent to the subject, the inert molecule or agent being capable of occupying the cyclodextrin.

81. The method of claim 80, wherein
(a) in the case of the 1:1 or 2:2 inclusion complex stoichiometry, a molar ratio between the cyclodextrin and the inert molecule or agent in the subject after administration of both is less than 1:1,
(b) in the case of the 1:2 inclusion complex stoichiometry, a molar ratio between the cyclodextrin and the inert molecule or agent in the subject after administration of both is less than 1:2, or
(c) in the case of the 2:1 inclusion complex stoichiometry, a molar ratio between the cyclodextrin and the inert molecule or agent in the subject after administration of both is less than 2:1.

82. The method of claim 81, wherein
(a) in the case of the 1:1 or 2:2 inclusion complex stoichiometry, a molar ratio between the cyclodextrin and the inert molecule or agent in the subject after administration of both is less than 2:1,
(b) in the case of the 1:2 inclusion complex stoichiometry, a molar ratio between the cyclodextrin and the inert molecule or agent in the subject after administration of both is less than 2:2, or
(c) in the case of the 2:1 inclusion complex stoichiometry, a molar ratio between the cyclodextrin and the inert molecule or agent in the subject after administration of both is less than 4:1.

83. The method of claim 81,
(a) in the case of the 1:1 or 2:2 inclusion complex stoichiometry, a molar ratio between the cyclodextrin and the inert molecule or agent in the subject after administration of both is less than 4:1,
(b) in the case of the 1:2 inclusion complex stoichiometry, a molar ratio between the cyclodextrin and the inert molecule or agent in the subject after administration of both is less than 4:2, or
(c) in the case of the 2:1 inclusion complex stoichiometry, a molar ratio between the cyclodextrin and the inert molecule or agent in the subject after administration of both is less than 8:1.

84. The method of claim 81, wherein
(a) in the case of the 1:1 or 2:2 inclusion complex stoichiometry, a molar ratio between the cyclodextrin and the inert molecule or agent in the subject after administration of both is less than 7:1,
(b) in the case of the 1:2 inclusion complex stoichiometry, a molar ratio between the cyclodextrin and the inert molecule or agent in the subject after administration of both is less than 7:2, or
(c) in the case of the 2:1 inclusion complex stoichiometry, a molar ratio between the cyclodextrin and the inert molecule or agent in the subject after administration of both is less than 14:1.

85. The method of claim 80, wherein the inert molecule or agent is proline.

86. The method of claim 85, wherein the inert agent is administered in combination with the cyclodextrin.

87. The method of claim 85, wherein the inert agent is administered prior to the subject receiving the cyclodextrin treatment.

88. The method of claim 85, wherein the inert agent is administered in composition with cyclodextrin.

89. The method of claim 85, wherein the inert agent is administered to the subject after the subject has received the cyclodextrin treatment.

90. The method of claim 85, wherein the cyclodextrin treatment is systemic cyclodextrin treatment.

91. The method of claim 85, wherein the cyclodextrin treatment is localized cyclodextrin treatment.

92. The method of claim 85, wherein the administration of the inert agent is via injection.

93. The method of claim 92, wherein the injection is perineural.

94. The method of claim 92, wherein the injection is intramuscular.

95. The method of claim 92, wherein the injection is subcutaneous.

96. The method of claim 92, wherein the injection is intradermal.

97. The method of claim 92, wherein the injection is intraarticular.

98. The method of claim 91, wherein the injection is intravenous.

99. The method of claim 80, wherein the administration of the inert agent is topical.

100. The method of claim 99, wherein the topical administration of the inert agent is via ear drops.

101. The method of claim 100, wherein the inert agent is administered together with a compound which increases delivery of the inert agent to the ear canal.

102. A method of treating spasticity in a subject in need of treatment, comprising:
injecting the patient with a composition comprising a majority unoccupied cyclodextrin locally to a motor nerve of a muscle spasticity site or source, the cyclodextrin being majority unoccupied with a guest compound.

103. The method of claim 102,
wherein the majority unoccupied cyclodextrin is a host,
wherein the compound is a guest, and
wherein
(a) in the case of a 1:1 or 2:2 inclusion complex stoichiometry, a molar ratio between the host and the guest is greater than 1:1,
(b) in the case of a 1:2 inclusion complex stoichiometry, the molar ratio between the host and the guest is greater than 1:2, or
(c) in the case of a 2:1 inclusion complex stoichiometry, the molar ratio between the host and the guest is greater than 2:1.

104. The method of claim 103,
(a) in the case of the 1:1 or 2:2 inclusion complex stoichiometry, a molar ratio between the host and the guest is greater than 2:1,
(b) in the case of the 1:2 inclusion complex stoichiometry, the molar ratio between the host and the guest is greater than 2:2, or
(c) in the case of the 2:1 inclusion complex stoichiometry, the molar ratio between the host and the guest is greater than 4:1.

105. The method of claim 103,
(a) in the case of the 1:1 or 2:2 inclusion complex stoichiometry, a molar ratio between the host and the guest is greater than 4:1,
(b) in the case of the 1:2 inclusion complex stoichiometry, the molar ratio between the host and the guest is greater than 4:2, or
(c) in the case of the 2:1 inclusion complex stoichiometry, the molar ratio between the host and the guest is greater than 8:1.

106. The method of claim 103, wherein
(a) in the case of the 1:1 or 2:2 inclusion complex stoichiometry, a molar ratio between the host and the guest is greater than 7:1,
(b) in the case of the 1:2 inclusion complex stoichiometry, the molar ratio between the host and the guest is greater than 7:2, or
(c) in the case of the 2:1 inclusion complex stoichiometry, the molar ratio between the host and the guest is greater than 14:1.

107. The method of claim 102, wherein the composition is substantially free of phenol.

108. The method of claim 102, wherein the majority unoccupied cyclodextrin is alpha, beta, or gamma cyclodextrin.

109. The method of claim 108, wherein the cyclodextrin is substituted alpha, beta, or gamma cyclodextrin.

110. The method of claim 109, wherein the substitution is a hydroxypropyl group.

111. The method of claim 109, wherein the substitution is a carboxymethyl group.

112. The method of claim 109, wherein the substitution is random methylation.

113. The method of claim 109, wherein the substitution is a sulfobutyl ether group.

114. The method of claim 102, wherein the majority unoccupied cyclodextrin is majority unoccupied hydroxypropyl cyclodextrin.

115. The method of claim 109, wherein the majority unoccupied hydroxypropyl cyclodextrin is majority unoccupied hydroxypropyl beta cyclodextrin.

116. The method of claim 109, wherein the majority unoccupied hydroxypropyl cyclodextrin is majority unoccupied hydroxypropyl alpha cyclodextrin.

117. The method of claim 109, wherein the majority unoccupied hydroxypropyl cyclodextrin is majority unoccupied hydroxypropyl gamma cyclodextrin.

118. The method of claim 102, wherein the composition is locally injected such that the composition is delivered to the proximity of a motor nerve

119. The method of claim 102, wherein the composition is locally injected such that the composition does not alter the function of a sensory nerve.

120. The method of claim 102,
wherein the composition comprises the majority unoccupied hydroxypropyl cyclodextrin at a concentration of greater than 13.33 mM, and
wherein 0.1 mL to 20 mL of the composition is injected.

121. The method of claim 120,
wherein the composition comprises the majority unoccupied hydroxypropyl cyclodextrin at a concentration of 100 mM to 200 mM.

122. The method of claim 102, wherein the composition consists essentially of the majority unoccupied hydroxypropyl cyclodextrin and a pharmaceutically acceptable carrier.

123. The method of claim 121, wherein the majority unoccupied hydroxypropyl cyclodextrin is majority unoccupied hydroxypropyl beta cyclodextrin and wherein the pharmaceutically acceptable carrier is saline, water for injection, phosphate buffered saline (PBS), or water.

124. The method of claim 102, wherein the injection is perineural.

125. The method of claim 102, wherein the injection is intramuscular.

126. The method of claim 102, wherein the injection is subcutaneous.

127. The method of claim 102, wherein the injection is intradermal.

128. The method of claim 102, wherein the injection is intravenous.

129. The method of claim 102, wherein the injection is intraarticular.

130. The method of claim 102, wherein the injection is repeated after at least 1 month.

131. The method of claim 102, wherein the injection is repeated after at least 2 months.

132. The method of claim 102, wherein the injection is repeated after at least 3 months.

133. The method of claim 102, wherein the injection is repeated after at least 4 months.

134. The method of claim 102, wherein the injection is repeated after at least 5 months.

135. The method of claim 102, wherein the injection is repeated after at least 6 months.
